(19) United States
(12) Patent Application Publication
(10) Pub. No.: US 2024/0268700 A1
(43) Pub. Date: Aug. 15, 2024

(54) SYSTEMS AND METHODS FOR MULTI-CONTRAST MULTI-SCALE VISION TRANSFORMERS

(71) Applicant: Subtle Medical, Inc., Menlo Park, CA (US)

(72) Inventors: Jiang LIU, Towson, MD (US); Gajanana Keshava DATTA, Los Altos, CA (US); Srivathsa Pasumarthi VENKATA, Sunnyvale, CA (US)

(21) Appl. No.: 18/636,423

(22) Filed: Apr. 16, 2024

Related U.S. Application Data
(63) Continuation of application No. PCT/US2022/048414, filed on Oct. 31, 2022.
(60) Provisional application No. 63/331,313, filed on Apr. 15, 2022.
(60) Provisional application No. 63/276,301, filed on Nov. 5, 2021.

(51) Int. Cl. A61B 5/055; G01R 33/50; G01R 33/56

(52) U.S. Cl. CPC A61B 5/055; G01R 33/50; G01R 33/5601

(57) ABSTRACT

Methods and systems are provided for synthesizing a contrast-weighted image in Magnetic resonance imaging (MRI). The method comprises: receiving a multi-contrast image of a subject, where the multi-contrast image comprises one or more images of one or more different contrasts; generating an input to a transformer model based at least in part on the multi-contrast image; and generating, by the transformer model, a synthesized image having a target contrast that is different from the one or more different contrasts of the one or more images, where the target contrast is specified in a query received by the transformer model.

### CROSS-REFERENCE TO RELATED APPLICATION

This application is a continuation of International Application No. PCT/US2022/048414 filed Oct. 31, 2022, which claims priority to U.S. Provisional Application No. 63/276,301 filed on Nov. 5, 2021, and U.S. Provisional Application No. 63/331,313 filed on Apr. 15, 2022, the content of which is incorporated herein in its entirety.

### STATEMENT AS TO FEDERALLY SPONSORED RESEARCH

This invention was made with government support under Grant No. R44EB027560) awarded by the National Institutes of Health. The government has certain rights in the invention.

### BACKGROUND

Magnetic resonance imaging (MRI) has been used to visualize different soft tissue characteristics by varying the sequence parameters such as the echo time and repetition time. Through such variations, the same anatomical region can be visualized under different contrast conditions and the collection of such images of a single subject is known as multi-contrast MRI. Multi-contrast MRI provides complimentary information about the underlying structure as each contrast highlights different anatomy or pathology. For instance, complementary information from multiple contrast-weighted images such as T1-weighted (T1), T2-weighted (T2), proton density (PD), diffusion weighted (DWI) or Fluid Attenuation by Inversion Recovery (FLAIR) in magnetic resonance imaging (MRI) has been used in clinical practice for disease diagnosis, treatment planning as well as down-steam image analysis tasks such as tumor segmentation. Each contrast provides complementary information. However, due to scan time limitations, image corruptions due to motion and artifacts, and different acquisition protocols, one or more of the multiple contrasts may be missing, unavailable or unusable. This poses a major challenge for the radiologists and the automated image analysis pipelines.

### SUMMARY

Currently, deep convolutional neural network (DCNN) based approaches such as missing data imputation have been proposed to tackle the problem of missing contrast, which aims to synthesize the missing contrast from existing contrasts. To fully utilize the available information for accurate synthesis, the conventional missing data imputation method takes all available contrast(s) as input to extract the complimentary information and output the missing contrast(s), which can be many-to-one, one-to-many, and many-to-many synthesis depending on the number of available contrasts. However, once trained, a DCNN model may only work with a fixed or predetermined number of input channels and combination of input contrasts (based on training data) lacking the capability of accommodating input data which may include any number or combination of input contrast. For example, in order to be able to handle any possible missing data scenario, it requires training (2^{P}−2) models, one for each possible input-output scenario, where P is the number of contrasts. Even some convolutional neural network (CNN) models may try to deal with multiple input combinations, due to inherent inductive bias of CNN models, such models are unable to capture and represent the intricate dependencies between the different input contrasts. For example, feature map fusion algorithm has been adopted to fuse the feature maps of input contrasts by a Max(·) function, such that the input to the decoder networks always has the same number of channels regardless of the number of input contrasts. However, the feature map fusion method has drawbacks where the input contrasts are encoded separately and the predefined Max(·) function does not necessarily capture the complimentary information within each contrast. In another example, pre-imputation method pre-imputes missing contrasts with zeros such that the input and output of synthesis networks always have P channels. However, such pre-imputation method also lacks the capability to capture the dependencies between the contrasts as it encourages the network to consider each input contrast independently instead of exploring complimentary information as any input channel can be zero. Further, current CNNs are not good at capturing the long range dependencies within the input images since they are based on local filtering, while spatially distant voxels in medical images can have strong correlations and provide useful information for synthesis. In addition, current CNNs are lack of interpretability, i.e., there is no explanation about why they produce a certain image and where the information comes from, which is crucial for building trustworthy medical imaging applications. Although several model interpretation techniques have been proposed for post-hoc interpretability analysis for CNN, they do not explain the reasoning process of how a network actually makes its decisions.

The present disclosure addresses the above drawbacks of the conventional imputation methods by providing a Multi-contrast and Multi-scale vision Transformer (MMT) for predicting missing contrasts. In some embodiments, the MMT may be trained to generate a sequence of missing contrasts based on a sequence of available contrasts. The MMT provided herein may be capable of taking any number and any combination of input sequences as input data and outputting/synthesizing a missing contrast. The output may be one or more missing contrasts. The method herein may beneficially provide flexibly that can handle a sequence of input contrasts and a sequence of output contrasts of arbitrary lengths to deal with exponentially many input-output scenarios with only one transformer model. Methods and systems herein may provide a vision transformer with a multi-contrast shifted windowing (Swin) scheme. In particular, the multi-contrast Swin transformer may comprise encoder and decoder blocks that may efficiently capture intra and inter-contrast dependencies for image synthesis with improved accuracy.

In some embodiments, the MMT based deep learning (DL) model may comprise a multi-contrast transformer encoder and a corresponding decoder that builds hierarchical representations of inputs and generates the outputs in a coarse-to-fine fashion. At test time or in the inference stage, the MMT model may take a learned target contrast query as input, and generate a final synthetic image as the output by reasoning about the relationship between the target contrasts and the input contrasts, and considering the local and global image context. For example, the MMT decoder may be trained to take a contrast query as an input and output the feature maps of the required (missing) contrast images.

In an aspect, methods and systems are provided for synthesizing a contrast-weighted image in Magnetic resonance imaging (MRI). Some embodiments of a computer-implemented method comprises: receiving a multi-contrast image of a subject, where the multi-contrast image comprises one or more images of one or more different contrasts: generating an input to a transformer model based at least in part on the multi-contrast image; and generating, by the transformer model, a synthesized image having a target contrast that is different from the one or more different contrasts of the one or more images, where the target contrast is specified in a query received by the transformer model.

In a related yet separate aspect, a non-transitory computer-readable storage medium including instructions that, when executed by one or more processors, cause the one or more processors to perform operations is provided. The operations comprise: receiving a multi-contrast image of a subject, where the multi-contrast image comprises one or more images of one or more different contrasts: generating an input to a transformer model based at least in part on the multi-contrast image; and generating, by the transformer model, a synthesized image having a target contrast that is different from the one or more different contrasts of the one or more images, where the target contrast is specified in a query received by the transformer model.

In some embodiments, the multi-contrast image is acquired using a magnetic resonance (MR) device, in some embodiments, the input to the transformer model comprises an image encoding generated by a convolutional neural network (CNN) model. In some cases, the image encoding is partitioned into image patches. In some cases, the input to the transformer model comprises a combination of the image encoding and a contrast encoding.

In some embodiments, the transformer model comprises: i) an encoder model receiving the input and outputting multiple representations of the input having multiple scales, ii) a decoder model receiving the query and the multiple representations of the input having the multiple scales and outputting the synthesized image. In some cases, the encoder model comprises a multi-contrast shifted window-based attention block. In some cases, the decoder model comprises a multi-contrast shifted window-based attention block. In some embodiments, the transformer model is trained utilizing a combination of synthesis loss, reconstruction loss and adversarial loss. In some embodiments, the transformer model is trained utilizing multi-scale discriminators. In some embodiments, the transformer model is capable of taking arbitrary number of contrasts as input.

In some embodiments, the method further comprises displaying interpretation of the transformer model generating the synthesized image. In some cases, the interpretation is generated based at least in part on attention scores outputted by a decoder of the transformer model. In some cases, the interpretation comprises quantitative analysis of a contribution or importance of each of the one or more different contrasts. In some cases, the interpretation comprises a visual representation of the attention scores indicative a relevance of a region in the one or more images or a contrast from the one or more different contrasts to the synthesized image.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically shows a multi-contrast multi-scale Transformer (MMT) network.

FIG. 1B shows an example of the architecture of a Multi-contrast Multi-scale vision Transformer (MMT), in accordance with some embodiments of the present disclosure.

FIG. 2A illustrates an example of the multi-contrast shifted window approach (M-Swin) for attention computation.

FIG. 2B shows an example of MMT encoder.

FIG. 3 shows an example of two consecutive multi-contrast shifted window (M-Swin) Transformer encoder blocks.

FIG. 4 shows an example of an MMT decoder.

FIG. 5 shows an example of the paired setup of the decoder blocks.

FIG. 6 shows an example of CNN image encoder and CNN image decoder.

FIGS. 7A-7D shows examples of qualitative performance (PSNR and SSIM) of the proposed model compared to CNN.

FIG. 8 shows example of qualitative performance of the model, in comparison to the ground truth images.

FIG. 9 shows an example of generating T1ce post-contrast images from a combination of T1, T2 and FLAIR images without requiring any contrast agent dose injection.

FIG. 10A shows the quantitative results of different methods on the test sets.

FIG. 10B shows an example of comparisons on the T1Gd synthesis task between the MMT model herein and other models.

FIG. 10C shows the detailed performance of MMT random models for all possible input combinations.

FIG. 10D shows the qualitative results of MMT-random model on the IXI dataset.

FIG. 10E shows the qualitative results of the MMT random model on the BraTS dataset.

FIG. 11 shows an example of attention score visualization.

FIG. 12 shows another example of interpretation of a model output.

FIG. 13 schematically illustrates a system implemented on an imaging platform for performing one or more methods/algorithms described herein.

FIG. 14 schematically shows an example of using CNN-based discriminators to guide the training of MMT for improving image quality.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Methods and systems herein may provide a deep learning-based algorithm for synthesizing a contrast-weighted image in Magnetic resonance imaging (MRI). Multi-contrast MRI provides complimentary information about the underlying structure as each contrast highlights different anatomy or pathology. By varying the sequence parameters such as the echo time and repetition time, the same anatomical region can be visualized under different contrast conditions and the collection of such images of a single subject is known as multi-contrast MRI. For example MRI can provide multiple contrast-weighted images using different pulse sequences and protocols (e.g., T1-weighted (T1), T2-weighted (T2), proton density (PD), diffusion weighted (DWI). Fluid Attenuation by Inversion Recovery (FLAIR) and the like in magnetic resonance imaging (MRI)). These different multiple contrast-weighted MR images may also be referred to as multi-contrast MR images. In some cases, one or more contrast-weighted images may be missing or not available. For example, in order to reduce scanning time, only selected contrasts are acquired while other contrasts are ignored. In another example, one or more of the multiple contrast images may have poor image quality that are not usable or lower quality due to reduced dose of contrast agent. It may be desirable to synthesize a missing contrast-weighted image based on other contrast images or to impute the missing data. The conventional missing data imputation method takes all available contrast(s) as input to extract the complimentary information and output the missing contrast(s), which can be many-to-one, one-to-many, and many-to-many synthesis depending on the number of available contrasts. However, once trained, a DCNN model may only work with a fixed or predetermined number of input channels and combination of input contrasts (based on training data) lacking the capability of accommodating input data which may include any number or combination of input contrast. Even some convolutional neural network (CNN) models may try to deal with multiple input combinations, due to inherent inductive bias of CNN models, such models are unable to capture and represent the intricate dependencies between the different input contrasts. Further, current CNNs are not good at capturing the long range dependencies within the input images since they are based on local filtering, while spatially distant voxels in medical images can have strong correlations and provide useful information for synthesis. In addition, current CNNs are lack of interpretability, i.e., there is no explanation about why they produce a certain image and where the information comes from, which is crucial for building trustworthy medical imaging applications. Although several model interpretation techniques have been proposed for post-hoc interpretability analysis for CNN, they do not explain the reasoning process of how a network actually makes its decisions.

The present disclosure provides a Multi-contrast and Multi-scale vision Transformer (MMT) for synthesizing a contrast image. The MMT herein may be capable of taking any number and combination of available contrast images as input and outputting/synthesizing any number of missing contrast(s). The term “available contrast” as utilized herein may generally refer to contrast images that have relatively high quality that are usable. The term “missing contrast” as utilized herein may refer to the contrast need to be synthesized due to various reasons such as low quality (not usable), or not available (e.g., not acquired). In some cases, the MMT may be trained to generate a sequence of missing contrasts based on a sequence of available contrasts of arbitrary lengths. This beneficially provides flexibility to deal with exponentially many input-output scenarios with only one model.

The multi-contrast multi-scale vision transformer (MMT) is provided for synthesis of any or different contrasts in MRI imaging. In some cases, the MMT model herein may be capable of replacing lower quality contrasts with synthesized higher quality contrasts without the need for rescanning. The MMT may be applied in a wide range of applications with any different combination of input contrasts and/or from images of different body parts. The provided MMT model may be applied to a variety of upstream and downstream applications and may achieve a variety of goals such as reducing scan time (e.g., by acquiring only certain contrasts while synthesizing the other contrasts), improving image quality (e.g., replacing a contrast with lower quality with the synthesized contrast), reducing the contrast agent dose (e.g., e.g., replacing a contrast image acquired with a reduced dose contrast agent with the synthesized contrast image), and any combination of the above or other applications.

In some cases, methods and systems herein may provide a vision transformer with a multi-contrast shifted windowing (Swin) scheme. In particular, the multi-contrast Swin transformer may comprise encoder and decoder blocks that can efficiently capture intra and inter-contrast dependencies for image synthesis with improved accuracy.

In some embodiments, the MMT based deep learning (DL) model may comprise a multi-contrast transformer encoder and a corresponding decoder that builds hierarchical representations of inputs and generates the outputs in a coarse-to-fine fashion. At test time or in the inference stage, the MMT model may take a learned target contrast query as input, and generate a final synthetic image as the output by reasoning about the relationship between the target contrasts and the input contrasts, and considering the local and global image context. For example, the MMT decoder may be trained to take a contrast query as an input and output the feature maps of the required (missing) contrast images. A contrast query may comprise learnable parameters that inform the decoder what contrast to synthesize (i.e., target contrast) and what information to decode from the encode output. Details about the contrast query and the MMT architecture are described later herein.

### Multi-Contrast and Multi-Scale Vision Transformer (MMT) Architecture

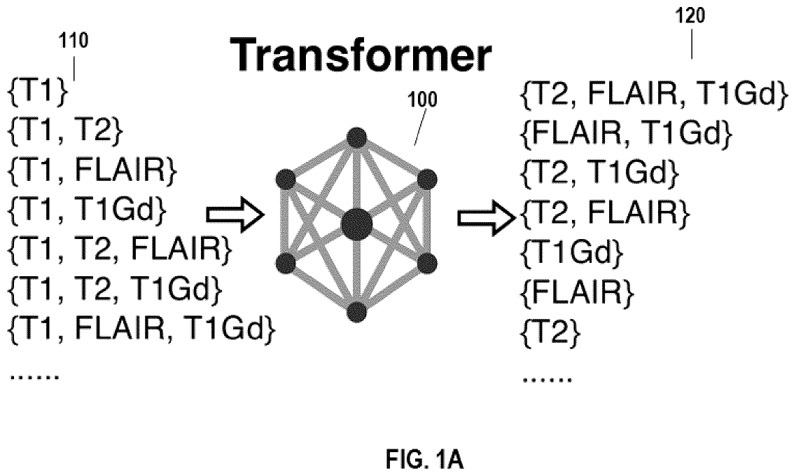

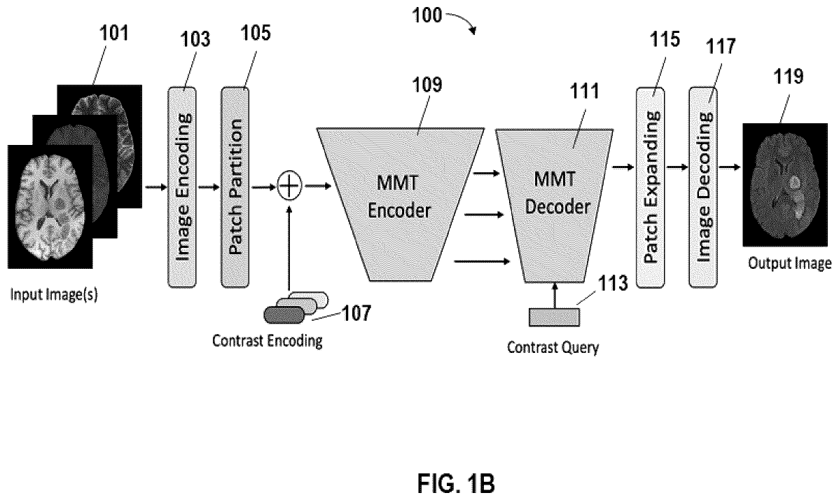

In an aspect, the present disclosure provides a Multi-contrast and Multi-scale vision Transformer (MMT) that is capable of taking any number and combination of input sequences and synthesizing a missing contrast. In particular, unlike the conventional data imputation method which usually has a fixed number of input channels or output channels (e.g., multiple input contrasts to generate one missing contrast, or one input contrast to generate one missing contrast, etc.), the MMT herein is capable of taking any number of contrast channels/images and output any number of missing contrast channels/images. As shown in FIG. 1A, the multi-contrast multi-scale Transformer (MMT) network 100 is capable of performing sequence-to-sequence prediction. As shown in the example, the input 110 may comprise a sequence formed with any number of images with different contrasts or any combination of contrast channels. For instance, the input sequence may be a sequence with one contrast such as {T1}, or sequence of multiple contrasts such as {T1, T2}, {T1, FLAIR}, {T1, T1Gd}, {T1, T2, FLAIR}, {T1, T2, T1Gd}, {T1, FLAIR, T1Gd}. The input sequence may comprise any available contrast images that are acquired using MR imaging device. The output may comprise the predicted (missing) contrast image. The output 120 may comprise a sequence including any number or combination of contrasts. For example, the output sequence corresponding to the input sequence {T1} may be {T2, FLAIR, T1Gd, FLAIR, T1Gd}. Similarly, the output sequences, {T2, T1Gd}, {T2, FLAIR, {T1Gd}, (FLAIR, {T2}, may include the missing contrasts complementing the input contrasts. For example, assuming MRI images of P contrasts x={x1, x2 . . . , xP} in total, given a sequence of arbitrary M (1≤M≤P−1) input contrasts x_{A}={xₐ_{ᵢ}}ᵢ₌₁^{M} the goal of MMT is to synthesize the remaining N contrasts x_{C}=x\x_{A}={x_{c}_{ᵢ}}ᵢ₌₁^{N}, where N=P−M, and A={aᵢ}^{M}ᵢ₌₁, C={cᵢ}^{N}ᵢ₌₁ are the indexes of available contrasts and missing contrasts respectively.

In some cases, the MMT may comprise an encoder Enc that first maps the input sequence x_{A} to a sequence of multi-scale feature representations f_{A}={fₐ_{ᵢ}}ᵢ₌₁^{M}, where fₐ_{ᵢ}=Enc(xₐ_{ᵢ})=[fₐ_{ᵢ}⁽¹⁾, fₐ_{ᵢ}⁽²⁾, . . . , fₐ_{ᵢ}^{(S)}], and fₐ_{ᵢ}^{(S)} is the feature of input contrast xₐ_{ᵢ} at scale s.

Given the mapping relationship f_{A} and the contrast queries q_{C}={q_{c}_{ᵢ}}ᵢ₌₁^{N} of the target contrasts, the MMT decoder Dec may reason about the input-target contrast relationship and synthesize the output sequence \hat{x}C={\hat{x}_{c}_{ᵢ}}ᵢ₌₁^{M}. In some cases, the output 120 may be generated one element at a time: \hat{x}_{c}_{ᵢ}=Dec(f_{A}; q_{c}_{ᵢ}), Cᵢ∈C.

The MMT may utilize a Shifting WINdow (swin) transformer that builds hierarchical feature maps by merging image patches in deeper layers thereby addressing the complexity of computing linear projections. The MMT may comprise multi-scale multi-contrast vision transformer for missing contrast synthesis. The MMT may comprise multi-contrast shifted window (M-Swin) based attention where attention computation is performed within local cross-contrast windows to model both intra- and inter-contrast dependencies for accurate image synthesis. The multi-scale multi-contrast vision transformer provided herein may improve over the conventional Shifting WINdow (swin) transformer with the capability to be applied to a wide range of data imputation and image synthesis tasks, particularly in medical imaging.

FIG. 1B shows an example of the architecture of Multi-contrast Multi-scale vision Transformer (MMT). In some embodiments, the input image 101 may comprise one or more different MRI contrast images (e.g. T1, T2, FLAIR, etc.). As described above, an input sequence may comprise images of any number of different contrasts or any combination of contrast channels. For instance, the input sequence may be a sequence with one contrast such as {T1}, or a sequence of multiple contrasts such as an input sequence {T1, T2}, an input sequence {T1, FLAIR}, an input sequence {T1, T1Gd}, an input sequence {T1, T2, FLAIR}, an input sequence {T1, T2. T1Gd}, or an input sequence {T1, FLAIR, T1Gd}. The one or more contrast images may be taken of the same body part.

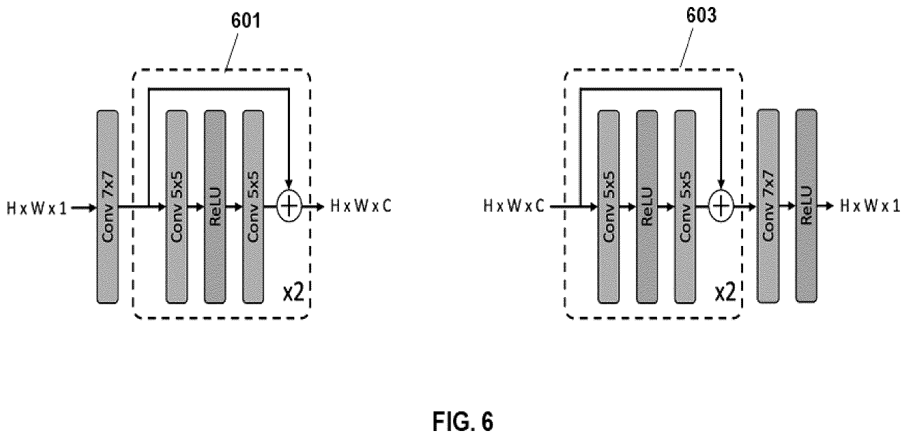

The input image(s) 101 may be passed through a series of convolutional neural network (CNN) encoders 103 to increase the receptive field of the overall network architecture. The CNN encoders may be small or shallow and may output a feature map representing the one or more input images. For example, the CNN encoders may have fewer number of parameters and/or layers. As an example, the small CNN used before the encoder and after the decoder may be shallow and have a number (e.g., 3, 4, 5, 6, 7, 8, etc.) of convolutional layers (with a ReLU activation in between). Details about the CNN encoder and decoder are described later herein with respect to FIG. 6.

Next, the feature map may be partitioned into small patches 105. Patch partitioning 105 may make the computation tractable which beneficially reduces the memory required for transformer models to perform matrix multiplication operations. The partitioned small patches may then be combined with the contrast encodings (e.g., T1, T2, FLAIR etc.) 107 and input to the MMT encoder 109. The contrast encodings may include vectors that encode information about a particular contrast. The contrast encodings inject contrast-specific information which helps the Transformer to be permutation-invariant to the input sequence. In some cases, the contrast encodings may include learnable parameters for each contrast in the input sequence and the target contrast. The learnable parameters may be learned during training process and the learnable parameters may represent the corresponding contrast. For example, the contrast encoding may be a n-dimensional vector including a plurality of 2D vectors each represents a contrast. When the 2D vectors are plotted in the 2D plane, vectors representing similar contrasts (e.g., T1 and T1Gd) lie closer and different contrasts (e.g., T1 and FLAIR) lie farther.

The MMT encoder 109 may generate feature maps at different levels/scales. For instance, the MMT encoder may map the input image(s) (e.g., sequence of contrast images) to a sequence of multi-scale feature representations. Details about the MMT encoder are described later herein. The features maps generated by the MMT encoder 109 may then be fed to the MMT decoder 111 to output patches of feature maps.

The MMT decoder 111 may work as a “virtual scanner” that generates the target contrast based on the encoder outputs and the corresponding contrast query 113. The MMT decoder 111 may be trained to take a contrast query 113 as an input and may output the feature maps of the required (missing) contrast image. The contrast queries may comprise vectors that initialize the decoding process for a given or target contrast. For example, a contrast query may be a 1×1×16C vector, 1×1×32C vector. 1×1×64C vector and the like. In some embodiments, the contrast queries are learnable parameters that inform the decoder what contrast to synthesize (e.g., what the missing/target contrast is) and what information to decode from the encoder outputs.

In some cases, the contrast queries 113 may be learned during training. The correspondence between a contrast query and a given contrast is learned during training. In some cases, the contrast queries are optimized during training, such that the decoder can generate high-quality images of a contrast when the corresponding contrast query is provided.

The decoder may combine the contrast query and encoder output for generating the queried contrast image. The feature maps may be upsampled by the “Patch Expanding” blocks 115 followed by an image decoder 117 to output the corresponding image(s) 119. The image decoder 117 may comprise a series of CNN decoders. In some cases, the series of CNN decoders 117 may be small or shallow CNN. Such MMT architecture 100 may be able to take any subset of input contrasts and synthesize one or more missing contrast images.

### Multi-Contrast Shifted Window Based Attention

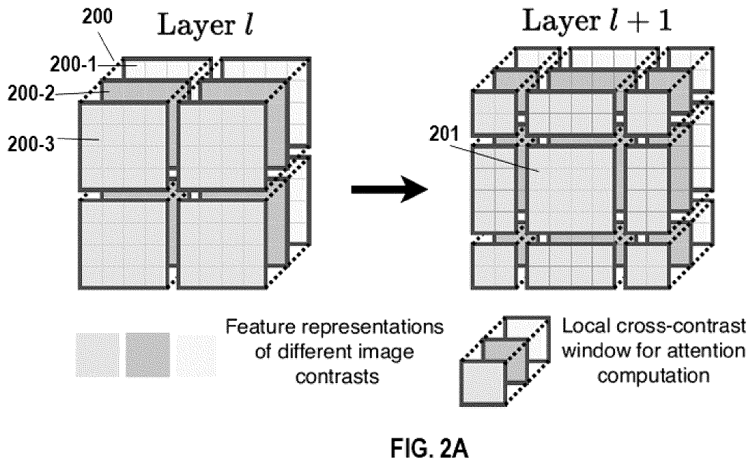

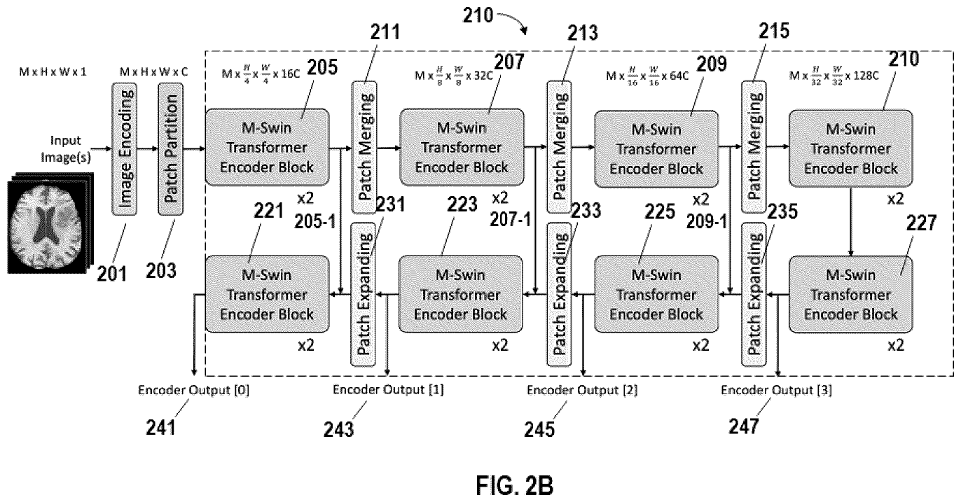

The MMT model herein may comprise multi-contrast shifted window (M-Swin) based attention where attention computation is performed within local cross-contrast windows to model both intra- and inter-contrast dependencies for accurate image synthesis. The MMT model herein may use shifted window partitioning in successive blocks to enable connections between neighboring non-overlapping windows in the previous layer. Compared to global computation, such local window based approach beneficially reduces computational complexity for synthesizing high resolution images as the complexity is quadratic with respect to the number of tokens. The M-Swin attention can be computed regardless of the number of contrasts. This beneficially allows the MMT to take any arbitrary subset of contrasts as input and generate the missing contrast(s) with only one model. FIG. 2A schematically illustrates an example of the multi-contrast shifted window approach (M-Swin) for attention computation. Attention is computed within local cross-contrast windows 200 to model inter- and intra-contrast dependencies. For example, in layer l, a regular window partitioning is used such that a local cross-contrast window 200 includes all the partitioned feature representations of the different image contrasts 200-1, 200-2, 200-3 in the local regular window. In the next layer l+1 the window partitioning is shifted (e.g., by shifting the local window 200).

### MMT Encoder

FIG. 2B shows an example of MMT encoder 210 of the MMT model herein. The MMT encoder 210 may be trained to generate hierarchical representations of input images at multiple scales. The MMT encode 210 may perform joint encoding of multi-contrast input to extract complimentary information for accurate synthesis. In some embodiments, the MMT encoder may be a U-Net or similar to U-Net. In some cases, the MMT encoder may have a paired set up of M-Swin transformer encoder blocks with Patch Merging in the downsample portion and Patch Expanding in the upsample portion. As shown in FIG. 2B, the MMT encoder may have a U-Net architecture and is trained to generate multi-scale representations of the input images.

In some embodiments, the MMT encoder 210 may perform joint encoding of multi-contrast input (i.e., input images of multiple contrast) to capture inter- and intra-contrast dependencies. The input image may comprise any number (e.g., M contrasts) of different contrast images. The M input image(s) may be processed by image encoding 201 and patch partition 203 and then supplied to the MMT encoder 210. The image encoding 201 and patch partition 203 can be the same as those described in FIG. 1. For example, the image encoding 201 may comprise a series of shallow CNNs. For instance, the M input images of size H×W are first passed through separate image encoding blocks 201 to project them to an arbitrary dimension (′. The patch partition layer 203 then splits each encoded image into non-overlapping patches and concatenates the features of each pixel. Each patch is considered as a “token” for attention computation. In the illustrated example, a patch size of 4×4 is used, which results in M× H/4×W/4 patch tokens of feature dimension 4×4×C=16C.

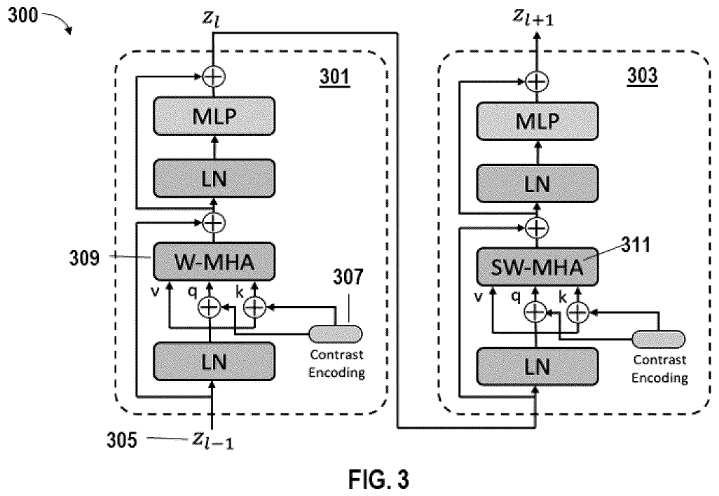

Next, a series of M-Swin encoder blocks are applied on the patch tokens to perform feature extraction. The MMT encoder 210 may comprise a downsampling portion or downsampling path. The downsampling portion/path of the MMT encoder may comprise a series of multi-contrast (M-Swin) transformer encoder blocks 205, 207, 209, 210. In some cases, a multi-contrast (M-Swin) transformer block 205, 207, 209, 210 may have a paired setup. For example, two successive M-Swin transformer encoder blocks may be paired (X2) and a pair 205, 207, 209 may be followed by a patch merging layer 211, 213, 215. In some cases, a plurality of pairs of M-Swin transformer encoder blocks may be followed by a patch merging layer. Details about the paired successive encoder blocks are described in FIG. 3.

In some cases, each pair of M-Swin transformer encoder blocks may be followed by a patch merging layer 211, 213, 215. The patch merging layer may be similar to a downsampling layer which reduces the spatial dimension of a feature map by a factor. For example, the patch merging layer concatenates the features of each group of 2×2 neighboring patches, and applies a linear layer on the concatenated features, which results in 2× reduction in spatial resolutions and 2× increase in feature dimensions. In the illustrated example, the patch merging layer reduces the spatial dimension of a feature map by a factor of 2. The reduction factor may or may not be the same across the multiple patch merging layers. As shown in the example, the output features of the first M-Swin Transformer encoder block 205 with size M×H/4×W/4×16C (M×height×width×channel, M is the number of input contrasts) is reduced to M×H/8×W/8×32C after first merger layer 211.

The MMT encoder may comprise an upsampling portion or upsampling path. The upsampling path of the MMT encoder may comprise a series of M-Swin transformer encoders 221, 223, 225, 227. In some cases, the series of M-Swin transformer encoders may also have a paired set-up where two successive encoder blocks may be followed by a patch expanding (or upsampling) layer 231, 233, 235. In the illustrated example, the patch expanding layer first applies a linear layer to increase the feature dimensions by a factor of two, and then each patch token is split into 2×2 neighboring tokens along the feature dimensions, which results in 2× increase in spatial resolutions and 2× reduction in feature dimensions. In some cases, the features 205-1, 207-1, 209-1 from the down-sampling path are concatenated with the up-sampled features produced by the patch expanding layers to reduce the loss of spatial information, and a linear layer is used to retain the same feature dimension as the up-sampled features.

At each stage of the up-sampling path, the MMT encoder may output the multi-scale representations of the input image(s) 241, 243, 245, 257. The multi-scale representations of the input image(s) may comprise representation of the input image(s) of various resolutions (e.g., H/4×W/4, H/8×W/8, H/16×W/16, H/32×W/32, etc.). The multi-scale representations of the input images(s) 241, 243, 245, 257 may be consumed by the MMT decoder in following steps. It should be noted that the MMT encoder and MMT decoder may comprise any number of M-Swin transformer encoder blocks and the M-Swin transformer encoder blocks may have variant configurations (e.g., every two or more pairs of M-Swin transformer encoder blocks are followed by one patch merging layer, etc.).

As described above, in some cases, the M-Swin transformer encoders of the MMT encoder may have a paired set-up. For example, a pair may be formed by two consecutive M-Swin Transformer encoder blocks. FIG. 3 shows an example 300 of two consecutive M-Swin Transformer encoder blocks. The first encoder block 301 takes the feature maps 305 from the previous layer as input and is passed through a LayerNorm (LN) layer. The output may be combined with the contrast encoding 307 and passed to a W-MHA (Window Multi-Head Attention) layer 309. The W-MHA may also be referred to as multi-contrast window based attention (MW-MHA) modules. Next, the output attention map is concatenated with the input feature map and is passed through a series of LN and MLP (multi-layer perceptron) layers. In some cases, MLP is a two-layer perceptron with GELU nonlinearity. Since Transformer is permutation-invariant to the input sequence, contrast encodings 307 is added to inject contrast-specific information, which are learnable parameters for each contrast. In some cases, relative position bias is also added in attention computation.

The second encoder block 303 may have a similar architecture except that it has a SW-MHA (Shifted Window Multi-Head Attention) layer 311 instead of a W-MHA layer. The SW-MHA may employ a multi-contrast shifted window based attention module as described above. In some cases, a local window of size Wₕ×W_{w} is extracted from the feature map of each contrast and a sequence of images M×Wₕ×W_{w} is formed for attention computation, where M is the number of input contrasts.

### MMT Decoder

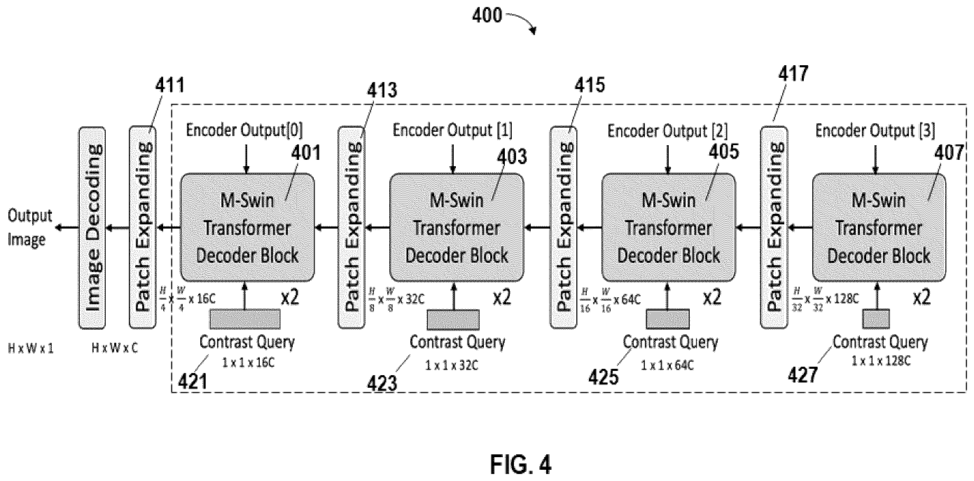

FIG. 4 shows an example of an MMT decoder 400. The MMT decoder may generate target output based on a contrast query. The MMT decoder functions as a “virtual scanner” that generates a target contrast based on the encoder outputs and the corresponding contrast query. As described above, the contrast queries may comprise vectors that initialize the decoding process for a given contrast. For example, a contrast query may be a 1×1×16C vector, 1×1×32C vector, 1×1×64C vector and the like. In some embodiments, the contrast queries are learnable parameters that inform the decoder what contrast to synthesize and what information to decode from the encoder outputs. In some cases, the contrast queries may be learned during training. The correspondence between a contrast query and a given contrast is learned during training. The contrast queries are optimized during training, such that the decoder can generate high-quality images of a contrast when the corresponding contrast query is provided.

The decoder blocks progressively decode the encoder outputs at different scales (e.g., multi-scale representations of the input image(s)) and generates the desired output. In some embodiments, the MMT decoder may generate the output image in a coarse-to-fine fashion, which allows it to consider both local and global image context for accurate image synthesis. In some embodiments, the MMT decoder may comprise a series of a M-Swin Transformer Decoder blocks. In some cases, the series of M-Swin Transformer Decoder blocks may be paired such that each pair 401, 403, 405, 407 may be followed by a patch expanding (upsampling) layer 411, 413, 415, 417. For example, the patch expanding layer first applies a linear layer to increase the feature dimensions by a factor of two, and then each patch token is split into 2×2 neighboring tokens along the feature dimensions, which results in increase in spatial resolutions by factor of 2 and reduction in feature dimensions by factor of 2. In some cases, each pair of M-Swin transformer decoder blocks 411, 413, 415, 417 may also take as input the learned contrast query of dimensions 421, 423, 425, 427 (e.g., 128C, 64C, 32C and 16C, where C is the number of channels of the feature map) respectively. In the illustrated example, the last patch merging layer performs a 4× up-sampling and restores the feature resolution to H×W by splitting each patch token into 4×4 neighboring tokens along the feature dimensions, which reduces the feature dimension from 16C to C.

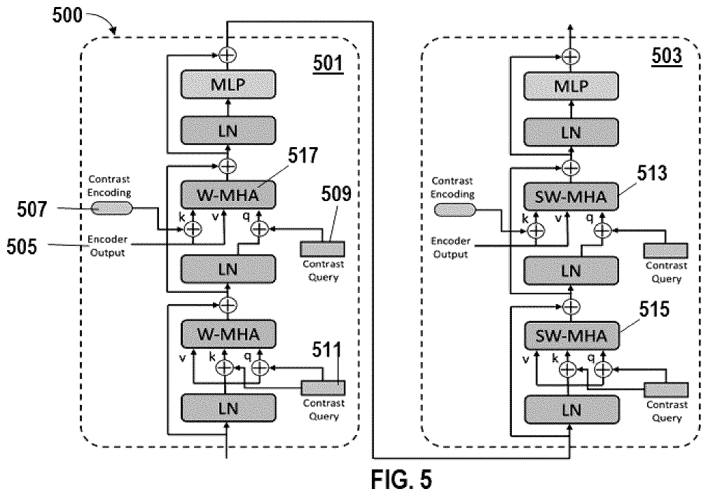

As described above, the MMT decoder may also have paired set up to the M-Swin Transformer Decoder blocks. FIG. 5 shows an example 500 of the paired setup of the decoder blocks. The M-Swin decoder block may have a similar structure as the encoder block, except that there is an additional SW-MHA layer that decodes the outputs of the MMT encoder. A first decoder block 501 may have two pairs of LN+W-MHA layers where the first layer takes the contrast query 511 as the input. The second W-MHA layer 517 takes as input, the corresponding encoder output 505 and the contrast encoding 507 in addition to the contrast query 509. The decoder block may have a LN+MLP combination. The second decoder block 503 may have a similar architecture, except that it may have SW-MHA (Shifted Window Multi-Head Attention) layers 513, 515 instead of W-MHA layers.

The additional W-MHA 517 or SW-MHA layer 513 takes the features of input contrasts as key k and value v, and the feature of targeted contrast as query q in attention computation. Such layer may compare the similarity between the input contrasts and target contrasts to compute the attention scores, and then aggregate the features from input contrasts to produce the features of target contrasts using the attention scores as weights. The attention scores in this layer beneficially provides a quantitative measurement of the amount of information flowing from different input contrasts and regions for synthesizing the output image, which makes MMT inherently interpretable. For example, the system provided herein provides visualization of the attention score analysis to aid the interpretation of the MMT.

### CNN Image Encoding Block and Decoding Block

CNNs have inductive biases and do not support mixed combinatorial inputs for contrast synthesis. However, CNNs are shown to be good at extracting image features as CNNs can have large receptive fields with less parameters and computation compared to Transformer. The present disclosure may provide a combination of a transformer and CNN hybrid model to benefit from both CNN and transformer model. In some cases, the CNN hybrid model herein may use shallow CNN blocks for image encoding before feeding the images into Transformer blocks in the MMT encoder, as well as for final image decoding in the MMT decoder. FIG. 6 shows an example of CNN image encoder (601) and CNN image decoder (603). In some cases, small CNN networks (e.g., shallow CNN blocks) may be used for image encoding and decoding, before and after the MMT encoder and decoder respectively. The small CNN networks with the shallow CNN blocks for image encoding and image decoding can be the same as the image encoding block 103 and image decoding block 117 as described in FIG. 1. In the illustrated architectures of the image encoding and decoding blocks, “Conv n×n” denotes a convolutional layer with kernel size n×n and “ReLU” denotes a ReLU nonlinearity layer. In some cases, separate encoding/decoding pathways may be used for different contrasts. For example, each contrast may have an individual encoding/decoding pathway.

### Training Method

In some embodiments, the present disclosure may use adversarial training in the form of a least-squared GAN (generative adversarial network). In some embodiments, to further improve the perceptual quality of the synthetic images, CNN-based discriminators may be used to adversarially train the MMT.

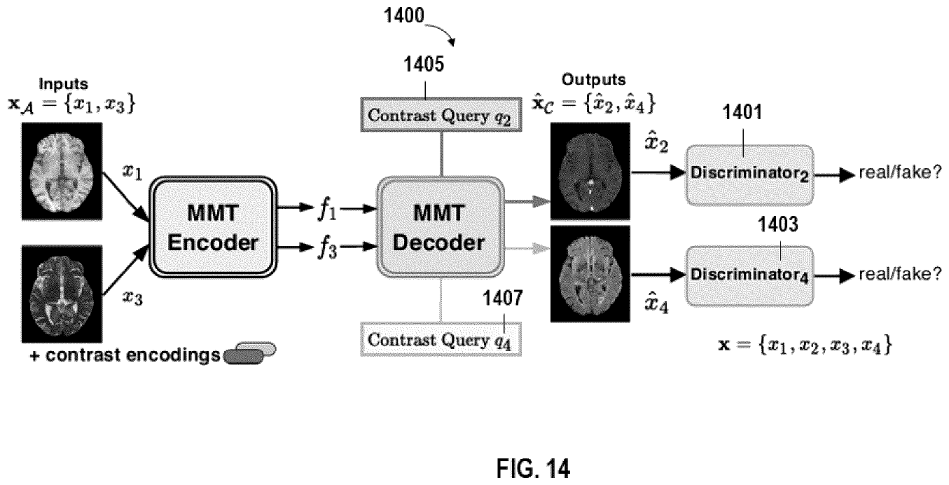

In some cases, multi-scale discriminators may be employed to guide MMT to produce both realistic details and correct global structure. FIG. 14 schematically shows an example 1400 of using CNN-based discriminators 1401, 1403 to guide the training of MMT for improving image quality. Separate discriminators (e.g., discriminator2 1401, discriminator4 1403) may be trained for each contrast (e.g., contrast query q2 1405, contrast query q4 1407) in order to learn contrast-specific features. This may beneficially further improve the perceptual quality of the synthesized missing contrasts.

In some embodiments, the training process may also comprise label smoothing to stabilize the training process. For example, instead of using binary values 0 or 1, the method herein may sample labels from uniform distributions. For example, fake labels Label_{f} may be drawn from a uniform distribution between 0 and 0.1 and real labels Labelᵣ may be drawn from a uniform distribution between 0.9 and 1.

$${Label}_{f} ∼ U ( 0 , 0.1 ) \\ {Label}_{r} ∼ U ( 0.9 , 1 )$$

Loss functions In some embodiments, the loss function for the model training may comprise a plurality of components including the synthesis loss, reconstruction loss and adversarial loss. Assume xⁱ the i-th input contrast, [symbol] the i-th reconstructed input contrast, y^{j} the j-th target contrast, and [symbol] the j-th output contrast (i=1, . . . , M, j=1, . . . , N), the loss function for the model training has three components as the following:

Synthesis Loss: Synthesis loss measures the pixel-wise similarity between output images and the ground-truth images. Synthesis loss trains MMT to accurately synthesize the missing contrasts when given the available contrasts. As an example, the synthesis loss may be defined as the L1 norm or the mean absolute difference between the output contrast and the target contrast. Following is an example of the synthesis loss:

$${ℒ}_{s} = \frac{1}{N} {\sum }_{j = 1}^{N} {\vert \vert {y}^{j} - \vert \vert}_{1}$$

Reconstruction Loss: MMT is expected to recover the input images when the decoder is queried with the contrast queries of input contrasts. This reconstruction loss component measures the ability of the network to reconstruct the inputs itself, which acts as a regularizer. It ensures the feature representations generated by the MMT encoder preserve the information in the inputs. As an example, the reconstruction loss is defined as the L1 distance between input images and reconstructed images. For example, the reconstruction loss is the L1 norm or the mean absolute difference between the input contrast and the reconstructed input contrast. Following is an example of the reconstruction loss:

$${ℒ}_{r} = \frac{1}{M} {\sum }_{i = 1}^{M} {\vert \vert {x}^{i} - \vert \vert}_{1}$$

[symbol] the i-th reconstructed input contrast which is generated by [symbol] =Dec(f_{A}; qᵢ), where qᵢ is the contrast queries of the input contrast.

Adversarial Loss: Adversarial loss encourages MMT to generate realistic images to fool the discriminators. Adversarial learning between the discriminators and MMT network forces the distribution of the synthetic images to match that of real images for each contrast. As an example, LSGAN is used as the objective. The adversarial loss may be defined as the squared sum of difference between the predicted and true labels for fake and real images. Dⱼ is the discriminator for the j-th output contrast, where Label_{f} and Labelᵣ are the labels for fake and real images respectively. Following is an example of the adversarial loss:

$${ℒ}_{adv} = \frac{1}{N} {\sum }_{j = 1}^{N} \{ {( {D}_{j} ( ) - {Label}_{f} )}^{2} + {( {D}_{j} ( {y}^{j} ) - {Label}_{r} )}^{2} \}$$

Overall Loss: The overall or total loss for the generator [symbol] _{G} is a weighted combination of the synthesis loss, reconstruction loss and the adversarial loss. Following is an example of the total loss:

$${ℒ}_{G} = {λ}_{r} {ℒ}_{r} + {λ}_{s} {ℒ}_{s} + {λ}_{adv} {ℒ}_{adv}$$

where values of the weights λᵣ, λₛ, λ_{adv} may be determined based on empirical data or dynamically determined based on training results. As an example, λᵣ is set to 5, Aₛ is set to 20 and λ_{adv} is set to 0.1.

The MMT model herein may support any combination of inputs and outputs for missing data imputation. By contrast, a conventional CNN based architecture may need separate models for each input combination. This significantly simplifies and improves the efficiency of model deployment in real-world clinical settings.

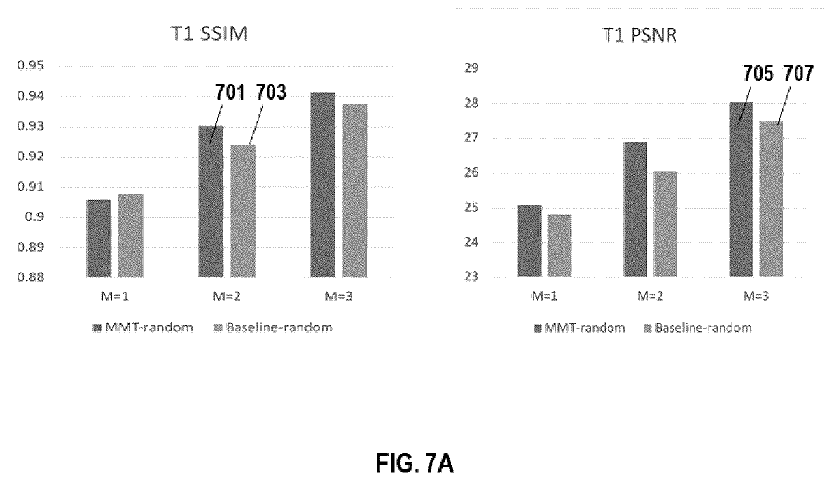

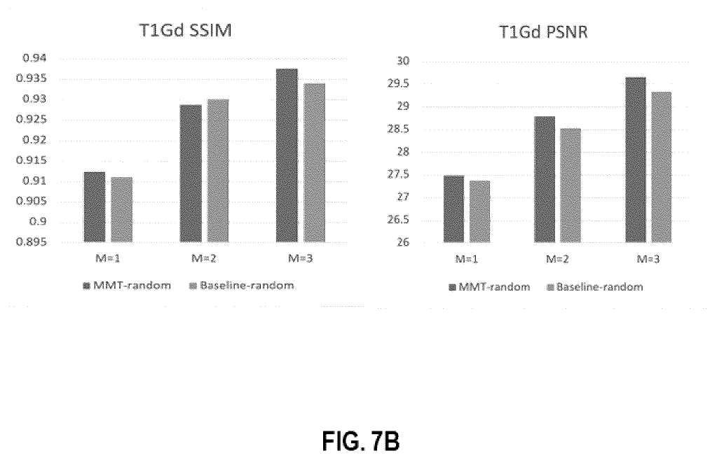

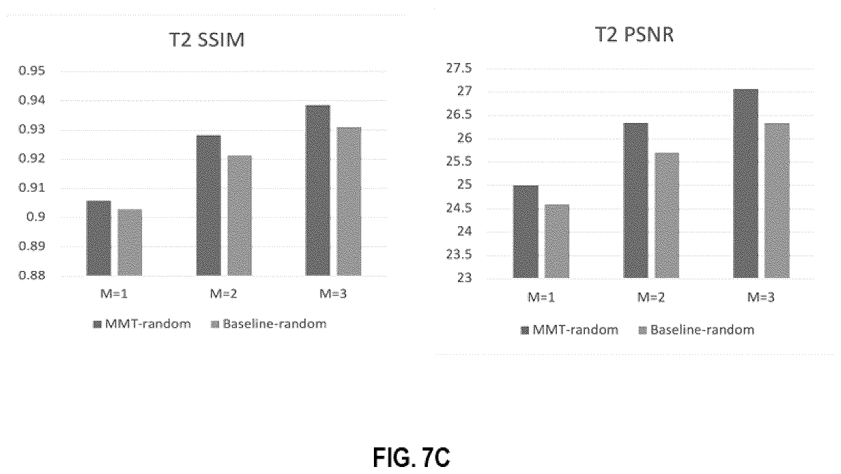

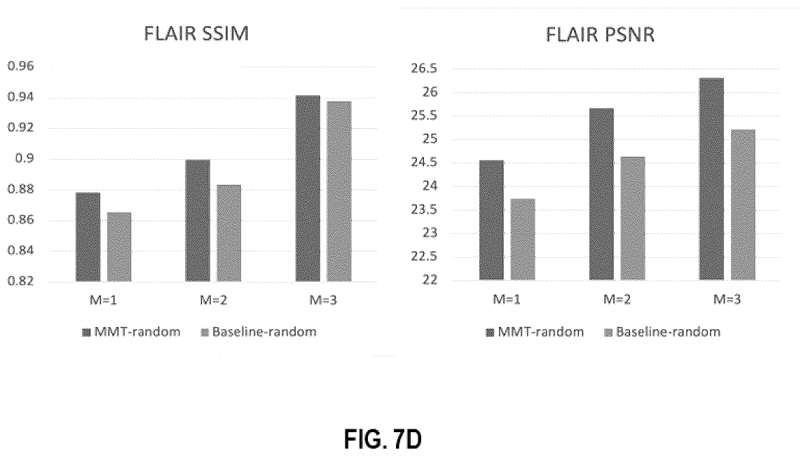

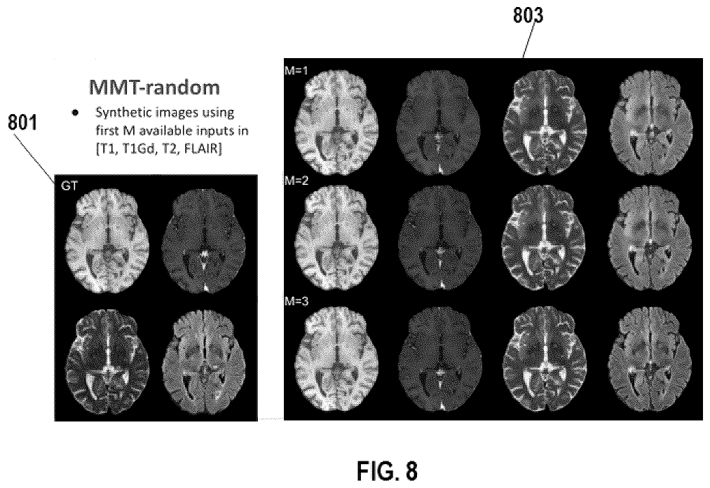

When compared to a CNN baseline, the proposed MMT model outperforms conventional models as measured by quantitative metrics. FIGS. 7A-7D shows examples of qualitative performance (PSNR and SSIM) of the proposed model compared to CNN. Superior quantitative metrics (PSNR and SSIM) of the proposed model (701, 705) in comparison to a CNN baseline (703, 707). M represents the number of missing contrasts. FIG. 8 shows example of qualitative performance of the model (803), in comparison to the ground truth images (801).

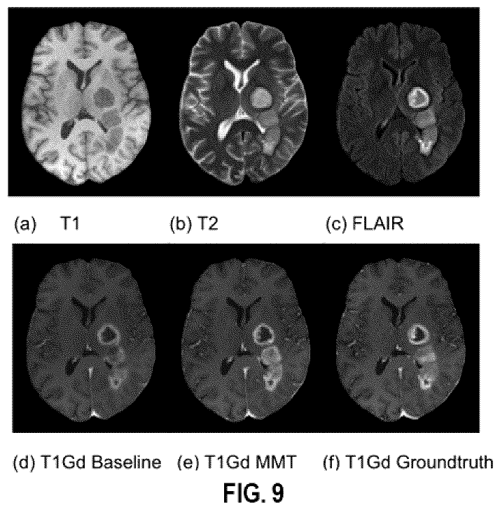

The provided MMT model may have various applications. For instance, the provided MMT model may be used as a contrast agent reduction synthesis model. The MMT model may be used to generate synthesized high quality contrast image to replace the low quality contrast image (due to contrast agent reduction). For example, the MMT model may be used as a Zero-Gd (Gadolinium) algorithm for Gadolinium (contrast agent) reduction. FIG. 9 shows an example of generating T1ce post-contrast images from a combination of T1, T2 and FLAIR images without requiring any contrast agent dose injection.

In other applications such as in any routine protocol, the provided MMT model may be capable of synthesizing complementary contrasts thus reducing the overall scan time by a significant amount. For example, in a L-Spine scanning protocol, the MMT model may generate the STIR (Short Tau inversion recovery) contrast from the T1 contrast and T2 contrast (i.e., T1-weighted and T2-weighted scans) thus saving the STIR sequence scanning time/procedure.

### Experiment and Examples

### Datasets

The models and methods herein are evaluated on multi-contrast brain MRI datasets: IXI and BraTS 2021. The IXI dataset consists of 577 scans from normal, healthy subjects with three contrasts: T1, T2 and PD-weighted (PD). The images were neither skull-stripped nor pre-registered. For each case, we co-registered the T1 and PD images to T2 using affine registration. In the experiments, 521, 28, and 28 cases were randomly selected for training, validation and testing respectively. The 90 middle axial slices are used and maintained the 256×256 image size. The BraTS 2021 (BraTS) dataset consists of 1251 patient scans with four contrasts: T1, post-contrast T1 weighted (T1Gd), T2-weighted (T2), and T2-FLAIR (FLAIR).

### Evaluation Settings

The models are evaluated using the peak signal-to-noise ratio (PSNR) and structural similarity index (SSIM), as well as LPIPS which captures perceptual similarity between images. The MMT provided herein is compared with two state-of-the art CNN methods for missing data imputation: MILR and MM-GAN. The comparison is performed for two scenarios: 1) single missing contrast, where only one contrast is missing, i.e., N=1; 2) random missing contrast, where N∈{1, 2, . . . , K−1} contrast(s) can be missing.

### Result

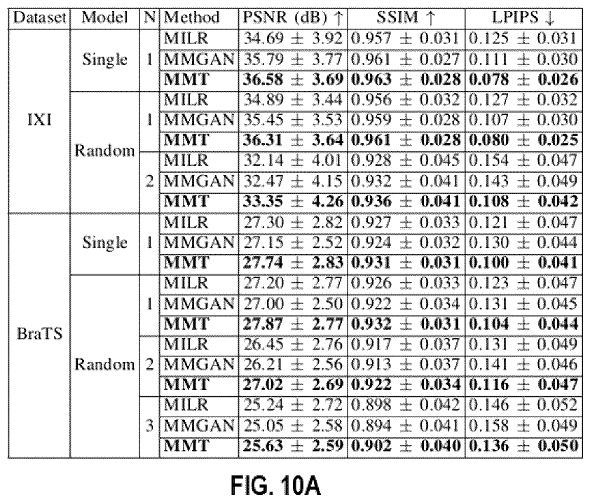

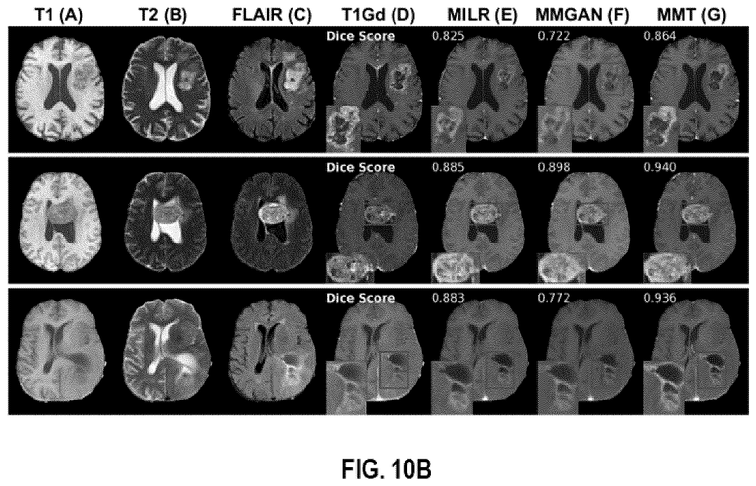

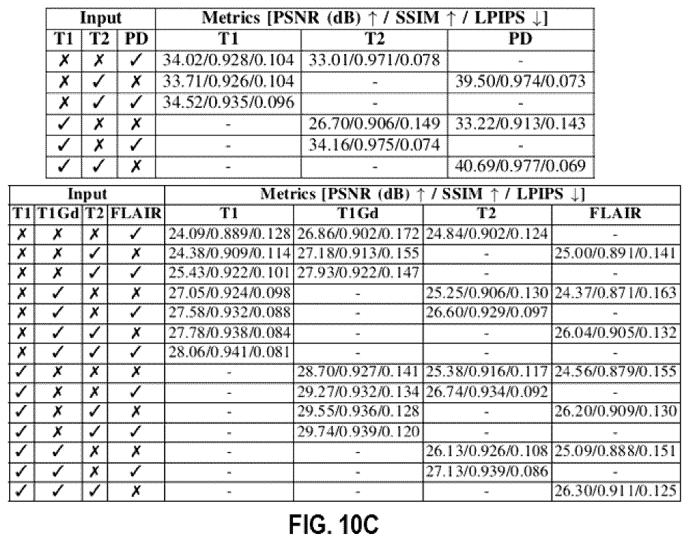

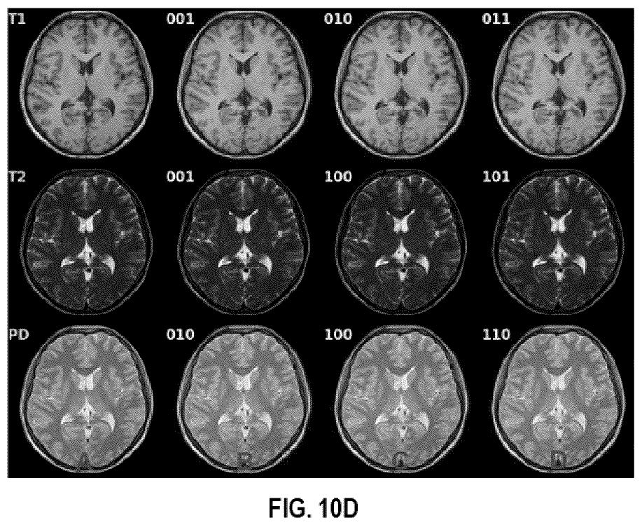

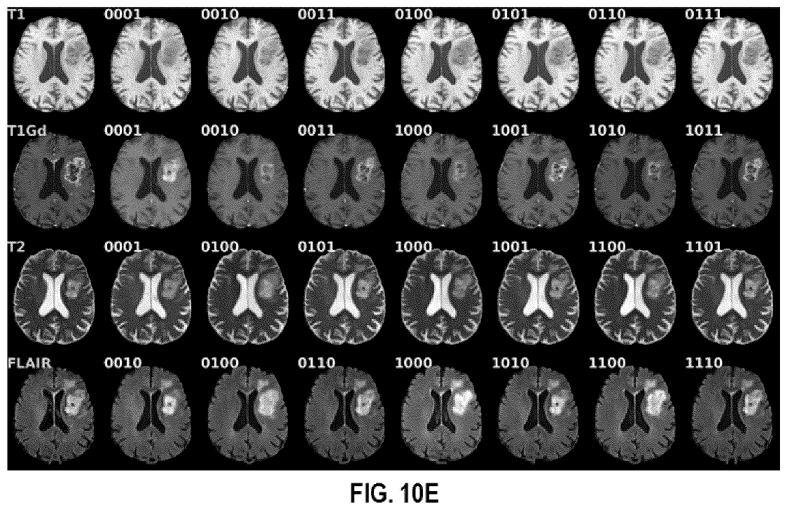

For each method and each dataset, two models are trained for the single and random missing contrast scenario respectively. Here, single model refer to the model trained for single missing contrast scenario and random models refer to the models trained in the random missing contrast scenario. FIG. 10A shows the quantitative results of different methods on the test sets. The up/down arrows indicates that higher/lower values correspond to better image quality respectively. N is the number of missing contrasts. The best performance is in bold with p<0.005. The results show that MMT are significantly better than MILR and MMGAN in all metrics and all scenarios on both IXI and BraTS datasets, based on Wilcoxon signed-rank test with p<0.005. The LPIPS scores of MMT are much lower than MILR and MMGAN, which indicates that the outputs of MMT have much better perceptual quality. A visual comparison of different single models on the T1Gd synthesis task (T1, T2, FLAIR→T1Gd) in FIG. 10B. This task is of great clinical value as synthesizing post-contrast T1 images from pre-contrast images can potentially reduce the cost of post-contrast MRI, avoid adverse reactions of contrast agents, and benefit the patients who are contraindicated to contrast agents. As shown in FIG. 10B, the outputs of MMT have better visual quality and more accurate synthesis of contrast enhancements. The enhancing tumor regions have sharper boundaries in MMT outputs compared to the other two methods. In addition, the MMT images achieves higher Dice scores when used for tumor segmentation. The example in FIG. 10B are comparisons on the T1Gd synthesis task on the BraTS dataset using the single models. Columns A-C are the input images. Column D is the ground truth T1Gd images. Columns E-G are the synthetic T1Gd images generated by different methods. The overlay in the box is the Tumor Core mask segmented on the respective images using an automatic tumor segmentation model. The Dicescore was computed between the masks generated on ground-truth images and synthetic images.

FIG. 10C shows the detailed performance of MMT random models for all possible input combinations. The upper table shows the quantitative performance of MMT random model on the IXI dataset for all input combinations. The lower table shows the quantitative performance of MMT random model on the BraTS dataset for all input combinations. FIG. 10D and FIG. 10E show the qualitative results. FIG. 10D shows the qualitative results of MMT-random model on the IXI dataset. In FIG. 10D. Column A shows the ground truth images: Column BD show the output images with input-output combination denoted by the binary string. The bits in the binary string are in the order [T1, T2, PD]. Bit value ‘0’/‘1’ means the contrast was missing/present during synthesis respectively. E.g., the binary string 101 in Row 2, Column D means the displayed T2 image was synthesized with T1 (bit 1) and PD (bit 3) as inputs. FIG. 10E shows the qualitative results of the MMT random model on the BraTS dataset. In FIG. 10E, Column A shows the ground truth images: Column B-H shows the output images with input-output combination denoted by the binary string. The bits in the binary string are in the order [T1, T1Gd, T2, FLAIR]. Bit value ‘0’/‘1’ means the contrast was missing/present during synthesis respectively. E.g., the binary string 1001 in Row 2, Column F means the displayed T1Gd image was synthesized with T1 (bit 1) and FLAIR (bit 4) as inputs. The same 1001 scenario is shown for T2 synthesis in Row 3, Column F. These results demonstrate that the MMT random models can reliably synthesize the missing contrasts across different input combinations. The synthesis performance for a particular contrast improves as more input contrasts are available, which indicates that MMT can effectively utilize the complementary information in the inputs for accurate synthesis.

### Interpretable Model

In another aspect of the present disclosure, the methods herein provide an interpretable MMT. Unlike the conventional interpretation method utilizing post-hoc explanation to explain the output of machine learning (ML) model, the MMT herein is inherently interpretable. The methods and systems herein may provide interpretation of the model in a quantitative manner with visual representation. As described above, the attention scores inside the MMT decoder indicate the amount of information coming from different input contrasts and regions for synthesizing the output, which makes MMT inherently interpretable.

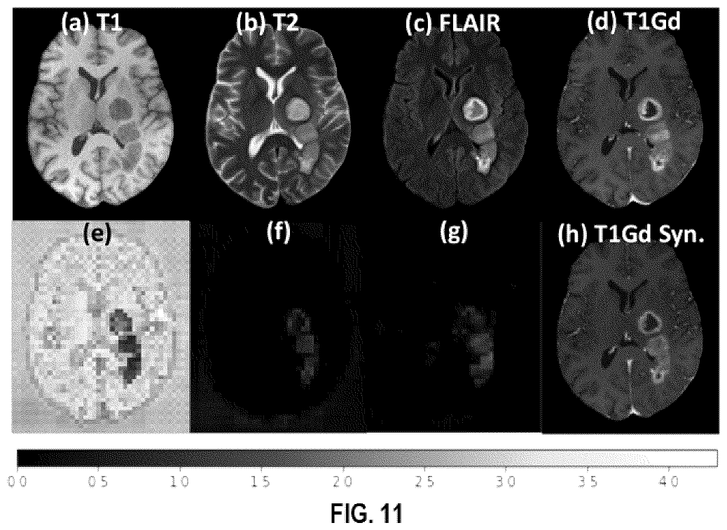

In some embodiments, the system herein provides visualization of interpretation of a model decision or reasoning. The visualization may be generated based on the attention scores. In some cases, the interpretation comprises a visual representation of the attention scores indicative a relevance of a region in the one or more images or a contrast from the one or more different input contrasts to the synthesized image. FIG. 11 shows an example of attention score visualization. In the example, panels (a)-(c) are input images; panel (d) is ground-truth T1Gd image, panels (e)-(g) are attention scores for the input contrasts from the last M-Swin decoder block and panel (h) is the output T1Gd image. The visualization of the attention score analysis provides interpretation about how a prediction or reasoning is made by the MMT (e.g., which region and/or which contrast contributes more or less to the prediction). The attention score may indicate a particular region within an input image and/or a particular contrast may have relatively more or less contribution to the synthesized output. For example, a higher attention score indicates more information coming from a particular region. The visualization in the example shows that to synthesize the T1Gd image, MMT mostly looks at the T1 input (e.g., higher attention score), which is reasonable since T1 and T1Gd are very similar except for the contrast enhancement in T1Gd. However, for the tumor region, MMT extracts more information from T2 and FLAIR as they provide stronger signals for the lesion. This visualization of attention score shows that the trained MMT understands the image context as well as the input-target contrast relationship, and attends to the right regions and contrasts for synthesis.

In addition to the above visualization, the attention scores may be used to interpret the model performance/output in various other ways. In some cases, methods herein may quantitatively measure the relative importance of each input contrast for a particular output by the percentage of attention scores. This beneficially allows for providing interpretation about which input image or portion of the input (e.g., a region in an image, a particular contrast, etc.) contributes to the predicted result and the extent of contribution. For example, for each input contrast, the method may comprise summing the attention scores over all MMT decoder blocks. In some cases, the method may further comprise normalizing the attention scores across input contrasts such that the sum is one to compute percentage of attention scores that each input holds. These percentages quantify the percentages of information coming from each input and therefore indicate their relative importance to the prediction.

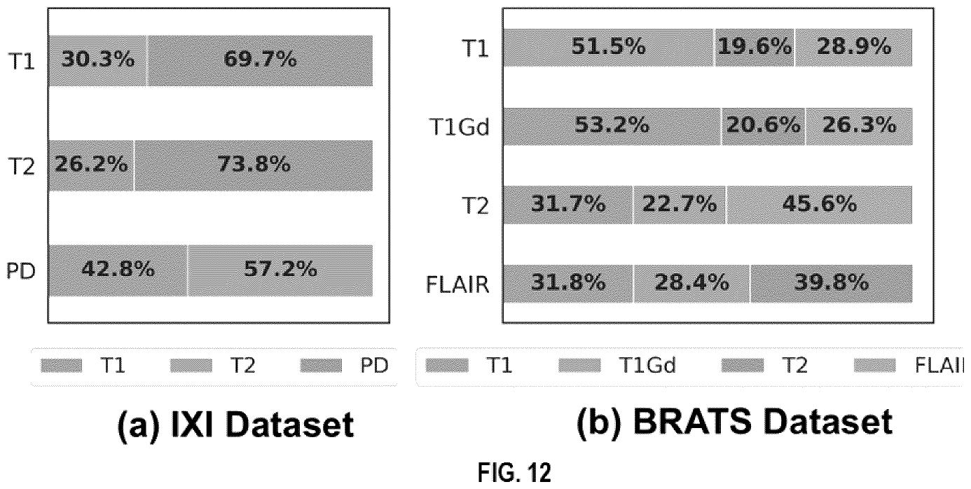

FIG. 12 shows an example of quantitative interpretation of a model output. In the example, the MMT single models are utilized and the attention scores are averaged on the test sets. On the IXI dataset, PD is the most important input for synthesizing T1 and T2, which contributes most of the information (˜70%). For synthesizing PD, T2 contributes more information than T1, which suggests higher similarity between T2 and PD. On the BraTS dataset. T1 and T1Gd are the most important input for each other contributing ˜50% of the information. The visual representation shows the MMT's prediction is reliable and reasonable since T1 and T1Gd are very similar except for the contrast enhancement in T1Gd. Similarly, T2 and FLAIR are the most important input for each other contributing ˜40% of the information.

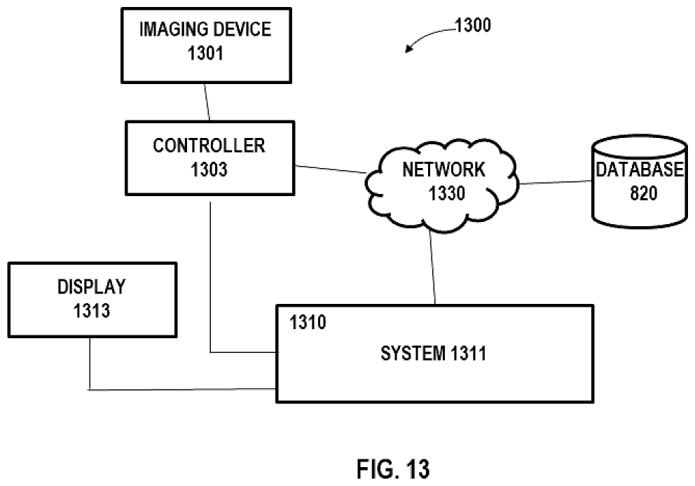

The systems and methods can be implemented on existing imaging systems without a need of a change of hardware infrastructure. In some embodiments, one or more functional modules such as the model interpretation visualization or MMT for missing contrast synthesis may be provided as separate or self-contained packages. Alternatively, the one or more functional modules may be provided as an integral system. FIG. 13 schematically illustrates a system 1311 implemented on an imaging platform 1300 for performing one or more methods/algorithms described herein. In some cases, the visualization of attention score (for interpreting model output) and/or the missing data imputation may be performed in real-time during image acquisition. Alternatively, one or more of the functions may be performed at any time post imaging or on-demand. The imaging platform 1300 may comprise a computer system 1310 and one or more databases 1320 operably coupled to a controller 1303 over the network 1330. The computer system 1310 may be used for implementing the methods and systems consistent with those described elsewhere herein to provide visualization of attention score and/or synthesizing the missing contrast(s), for example. The computer system 1310 may be used for implementing the system 1311. The system 1311 may include one or more functional modules such as a missing data imputing module comprising the MMT and/or a visualization module for model output interpretation. The functional modules may be configured to execute programs to implement the MMT for predicting the missing contrast(s) and/or generating the visualization of the attention scores as described elsewhere herein. Although the illustrated diagram shows the controller and computer system as separate components, the controller and computer system (at least some components of the system) can be integrated into a single component.

The system 1311 may comprise or be coupled to a user interface. The user interface may be configured to receive user input and output information to a user. The user interface may output a synthesized image of missing contrast generated by the system, for example, in real-time. In another example, the user interface may present to a user the visualization of the attention scores on the user interface. In some cases, additional explanation based on the attention score may be displayed. For example, user may be presented information related to whether the output generated by the MMT is reasonable or not. In some cases, the user input may be interacting with the visualization of the attention score. In some cases, the user input may be related to controlling or setting up an image acquisition scheme. For example, the user input may indicate scan duration (e.g., the min/bed) for each acquisition, sequence, ROI or scan time for a frame that determines one or more acquisition parameters for an acquisition scheme. The user interface may include a screen 1313 such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

In some cases, the user interface may comprise a graphical user interface (GUI) allowing a user to select a format for visualization of the attention score, view the explanation of the model output, view the synthesized image, and various other information generated based on the synthesized missing data. In some cases, the graphical user interface (GUI) or user interface may be provided on a display 1313. The display may or may not be a touchscreen. The display may be a light-emitting diode (LED) screen, organic light-emitting diode (OLED) screen, liquid crystal display (LCD) screen, plasma screen, or any other type of screen. The display may be configured to show a user interface (UI) or a graphical user interface (GUI) rendered through an application (e.g., via an application programming interface (API) executed on the local computer system or on the cloud). The display may be on a user device, or a display of the imaging system.

The imaging device 1301 may acquire image frames using any suitable imaging modalities live video or image frames may be streamed in using any medical imaging modality such as but not limited to MRI. CT, fMRI, SPECT. PET, ultrasound, etc. The acquired images may have missing data (e.g., due to corruption, degradation, low quality, limited scan time, etc.) such that the images may be processed by the system 1311 to generate the missing data.

The controller 1303 may be in communication with the imaging device 1301, one or more displays 1313 and the system 1311. For example, the controller 1303 may be operated to provide the controller information to manage the operations of the imaging system, according to installed software programs. In some cases, the controller 1303 may be coupled to the system to adjust the one or more operation parameters of the imaging device based on a user input.

The controller 1303 may comprise or be coupled to an operator console which can include input devices (e.g., keyboard) and control panel and a display. For example, the controller may have input/output ports connected to a display, keyboard and other I/O devices. In some cases, the operator console may communicate through the network with a computer system that enables an operator to control the production and display of live video or images on a screen of display. In some cases, the image frames displayed on the display may be generated by the system 1311 (e.g., synthesized missing contrast image(s)) or processed by the system 1311 and have improved quality.

The system 1311 may comprise multiple components as described above. In addition to the MMT for missing data imputation and the model output interpretation module, the system may also comprise a training module configured to develop and train a deep learning framework using training datasets as described above. The training module may train the plurality of deep learning models individually. Alternatively or in addition to, the plurality of deep learning models may be trained as an integral model. In some cases, the training module may be configured to generate and manage training datasets.

The computer system 1310 may be programmed or otherwise configured to implement the one or more components of the system 1311. The computer system 1310 may be programmed to implement methods consistent with the disclosure herein.

The imaging platform 1300 may comprise computer systems 1310 and database systems 1320, which may interact with the system 1311. The computer system may comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The computer system 1310 can communicate with one or more remote computer systems through the network 1330. For instance, the computer system 1310 can communicate with a remote computer system of a user or a participating platform (e.g., operator). Examples of remote computer systems include personal computers (e.g., portable PC), slate or tablet PC's (e.g., Apple® iPad, Samsung® Galaxy Tab), telephones. Smart phones (e.g., Apple® iPhone, Android-enabled device, Blackberry®), or personal digital assistants. The user can access the computer system 1310 or the system via the network 1330.

The imaging platform 1300 may comprise one or more databases 1320. The one or more databases 1320 may utilize any suitable database techniques. For instance, structured query language (SQL) or “NoSQL” database may be utilized for storing image data, collected raw data, attention scores, model output, enhanced image data, training datasets, trained model (e.g., hyper parameters), user specified parameters (e.g., window size), etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes: they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present disclosure. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network 1330 may establish connections among the components in the imaging platform and a connection of the imaging system to external systems. The network 1330 may comprise any combination of local area and/or wide area networks using both wireless and/or wired communication systems. For example, the network 1330 may include the Internet, as well as mobile telephone networks. In one embodiment, the network 1330 uses standard communications technologies and/or protocols. Hence, the network 1330 may include links using technologies such as Ethernet, 802.11, worldwide interoperability for microwave access (WiMAX), 2G/3G/4G/5G mobile communications protocols, asynchronous transfer mode (ATM), InfiniBand, PCI Express Advanced Switching, etc. Other networking protocols used on the network 1330 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS). Internet Protocol security (IPsec), etc. In another embodiment, the entities on the network can use custom and/or dedicated data communications technologies instead of, or in addition to, the ones described above.

The missing data imputation methods or system herein may comprise any one or more of the abovementioned features, mechanisms and components or a combination thereof. Any one of the aforementioned components or mechanisms can be combined with any other components. The one or more of the abovementioned features, mechanisms and components can be implemented as a standalone component or implemented as an integral component.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than.” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for synthesizing a contrast-weighted image comprising:
(a) receiving a multi-contrast image of a subject, wherein the multi-contrast image comprises one or more images of one or more different contrasts;
(b) generating an input to a transformer model based at least in part on the multi-contrast image; and
(c) generating, by the transformer model, a synthesized image having a target contrast that is different from the one or more different contrasts of the one or more images, wherein the target contrast is specified in a query received by the transformer model.

2. The computer-implemented method of claim 1, wherein the multi-contrast image is acquired using a magnetic resonance (MR) device.

3. The computer-implemented method of claim 1, wherein the input to the transformer model comprises an image encoding generated by a convolutional neural network (CNN) model.

4. The computer-implemented method of claim 3, wherein the image encoding is partitioned into image patches.

5. The computer-implemented method of claim 3, wherein the input to the transformer model comprises a combination of the image encoding and a contrast encoding.

6. The computer-implemented method of claim 1, wherein the transformer model comprises: i) an encoder model receiving the input and outputting multiple representations of the input having multiple scales, ii) a decoder model receiving the query and the multiple representations of the input having the multiple scales and outputting the synthesized image.

7. The computer-implemented method of claim 6, wherein the encoder model comprises a multi-contrast shifted window-based attention block.

8. The computer-implemented method of claim 6, wherein the decoder model comprises a multi-contrast shifted window-based attention block.

9. The computer-implemented method of claim 1, wherein the transformer model is trained utilizing a combination of synthesis loss, reconstruction loss and adversarial loss.

10. The computer-implemented method of claim 1, wherein the transformer model is trained utilizing multi-scale discriminators.

11. The computer-implemented method of claim 1, wherein the transformer model is capable of taking arbitrary number of contrasts as input.

12. The computer-implemented method of claim 1, further comprising displaying interpretation of the transformer model generating the synthesized image.

13. The computer-implemented method of claim 12, wherein the interpretation is generated based at least in part on attention scores outputted by a decoder of the transformer model.

14. The computer-implemented method of claim 12, wherein the interpretation comprises quantitative analysis of a contribution or importance of each of the one or more different contrasts.

15. The computer-implemented method of claim 12, wherein the interpretation comprises a visual representation of the attention scores indicative a relevance of a region in the one or more images or a contrast from the one or more different contrasts to the synthesized image.

16. A non-transitory computer-readable storage medium including instructions that, when executed by one or more processors, cause the one or more processors to perform operations comprising:
(a) receiving a multi-contrast image of a subject, wherein the multi-contrast image comprises one or more images of one or more different contrasts;
(b) generating an input to a transformer model based at least in part on the multi-contrast image; and
(c) generating, by the transformer model, a synthesized image having a target contrast that is different from the one or more different contrasts of the one or more images, wherein the target contrast is specified in a query received by the transformer model.

17. The non-transitory computer-readable storage medium of claim 16, wherein the multi-contrast image is acquired using a magnetic resonance (MR) device.

18. The non-transitory computer-readable storage medium of claim 16, wherein the input to the transformer model comprises an image encoding generated by a convolutional neural network (CNN) model.

19. The non-transitory computer-readable storage medium of claim 18, wherein the image encoding is partitioned into image patches.

20. The non-transitory computer-readable storage medium of claim 18, wherein the input to the transformer model comprises a combination of the image encoding and a contrast encoding.
